(19) United States
(12) Patent Application Publication
(10) Pub. No.: US 2023/0115919 A1
(43) Pub. Date: Apr. 13, 2023

(54) INSTRUMENT CONNECTION DEVICE AND METHOD FOR PRODUCTION THEREOF

(71) Applicant: Erbe Elektromedizin GmbH, Tuebingen (DE)

(72) Inventors: Johannes Heym, Tuebingen (DE); Elmar Ahlburg, Trochtelfingen (DE)

(73) Assignee: Erbe Elektromedizin GmbH, Tuebingen (DE)

(21) Appl. No.: 17/955,025

(22) Filed: Sep. 28, 2022

(30) Foreign Application Priority Data
Oct. 8, 2021 (EP) 21201714.9

(51) Int. Cl. A61M 39/12

(52) U.S. Cl. CPC A61M 39/12; A61M 2039/1027

(57) ABSTRACT

An instrument connection device having a flexible hose-like body with a flexible wall in which the proximal end of a hose is inserted. The hose has a lumen that extends longitudinally through the hose. A wire is arranged inside the hose, for example, inside the lumen. The wire is guided through a punctured hole in the wall that the wire may create during a penetration of wall. The punctured hole may be straight and guides the obliquely. For example, the hole may be inclined to the radial axis and the longitudinal center axis.

### CROSS-REFERENCE TO RELATED APPLICATION

This application claims priority to European Application No. 21201714.9, filed Oct. 8, 2021, the entirety of which is incorporated by reference herein.

### TECHNICAL FIELD

Embodiments of the invention described herein relate to an instrument connection device, particularly for instruments that require a gas supply and current supply. Embodiments of the invention also relate to a method for production of such an instrument connection device.

### BACKGROUND

Surgical instruments for treatment of human or animal bodies are known that require a gas supply as well as a current supply for operation. For example, U.S. Pat. No. 7,717,911 B2 discloses for this purpose an instrument configured as flexible probe that substantially consists of a long hose through the lumen of which a wire extends. At the distal end of the hose an end of the wire is held approximately centrally in the hose and in this manner defines an electrode. During operation a radio frequency alternating voltage is applied to the wire and argon flows through the lumen of the hose. A plasma jet is created at the distal end of the probe.

For connection of such instruments to a supply apparatus EP 1 515 659 B1 describes a connector having a housing in which the proximal end of the probe is located. The housing is connected to a gas junction that comprises a gas channeling lumen that connects the hose with a filter. In addition, the gas junction guides an electrical line out of the lumen of the hose and thereby seals the lumen of the hose to the exterior.

This instrument connection device has been proven in general, however requires a remarkable manufacturing effort.

### SUMMARY

It is the object of embodiments of the invention to provide an instrument connection device that can be simply manufactured and also remains reliable for the long term.

The instrument connection device according to embodiments of the invention comprises a gas junction having a hollow cylindrical body with a flexible wall being part thereof. The wall limits a through channel that is defined concentrically to a longitudinal or axial direction. This longitudinal axis is the center axis of the hollow cylindrical body. In the wall of the hollow cylindrical body a punctured hole is provided through which a wire extends. This wire extends in the following inside a hose that can be a connection hose for the instrument or alternatively also part of the instrument. The wire can thereby extend through the lumen of the hose or can be completely or partly, e.g., in sections, embedded in the hose material. In addition, the wire can be arranged axially (longitudinally) and, if desired, also transversely movable inside the lumen of the hose or alternatively, can be arranged axially immovably inside the hose.

The hose is arranged inside the through channel of the flexible hollow cylindrical body in abutment against the wall thereof, such that a gas-tight connection between the hollow cylindrical body and the hose is created. Preferably the wall of the body abuts under pretension against the hose. Particularly, connections are considered to be gas tight, if at a pressure difference of at least 500 mbar no gas flow occurs along the wire through the hose wall. Preferably the wall thickness of the body and its material characteristics are in terms of material selection and elasticity particularly such that also higher pressure differences, of e.g. 1, 2, 3 or 4 bar do not result in a gas flow along the wire through the hose wall.

The punctured hole is a hole created by material displacement. Preferably the punctured hole is exclusively created by material displacement without material removal. It distinguishes in so far from a bore or a punched hole, which is created by ablation or removal of material. On the contrary, the punctured hole formed in the flexible wall according to an embodiment of the invention is elastically widened by the wire, such that the flexible wall of the gas junction is in close contact with the wire in a sealing manner. Preferably the flexible wall abuts against the wire elastically pretensioned at the punctured hole. In other words, the flexible wall is under an elastic pretension at the punctured hole. The punctured hole has the tendency to close if the wire is removed. For this reason, it is possible to guide the wire through the punctured hole without a gasket. Neither inside the through channel nor outside the elastic body sealing material on the wire is necessary. The elastic body itself seals on the wire. However, on the wire and on the outside of the elastic body, where the wire leaves the elastic body, sealing material can be attached, e.g., in form of adhesive or the like. This is however only an option and not required in many cases.

In an embodiment of the invention, the wire may be metallic bare wire, i.e., without a non-metallic surface coating. However, in other embodiments, the wire can be provided with a coating made of plastic, for example, that tightly adheres to its surface. Independent therefrom it is possible to heat the wire after penetration through the wall to melt it to the wall. If a non-metallic meltable coating is provided on the wire, it can be melted at least locally, e.g., by means of energy influence, by radiation, heat, ultrasound or the like, to create an additional sealing and/or adhesive bond with the material of the wall.

Preferably the punctured hole is orientated obliquely to the wall. In doing so, the wire also extends obliquely to the wall and thus also obliquely to the longitudinal axis of the elastic body through the wall. By means of these measures, i.e., by means of an extension of the punctured hole and the wire deviating from the radial direction, the contact surface between the wire and the wall of the elastic body is maximized, which supports the sealing effect.

The angle between the longitudinal direction of the through channel and the punctured hole or the wire guided through the punctured hole can vary and can be higher than 10°, higher than 20°, higher than 30° or also higher than 40°. The aforementioned angle should be less than or equal to 90°, better less than 80° or, as it is preferred, less than 70°. It is particularly preferred if the angle is less than 60°. This leads to a gas-tight wire passage that can be simply manufactured and is long-term reliable.

In a preferred embodiment, the body has a flexibility that is higher than the flexibility of the hose such that the flexible body gets in tight contact with the hose in a sealing manner. The hose is slightly bending-resistant due to its lower flexibility and thus can be inserted into the through channel of the flexible body. In this manner the inner diameter of the through channel can be (slightly) less than the outer diameter of the hose. The hose is held by friction-fit inside the through channel after insertion. For supporting the leak tightness and the friction-fit, a clamping device can be provided in the instrument connection device that biases the elastic body locally radially inward and thereby increases the pressing between the elastic body and the hose.

The elastic body can consist of a silicone plastic. The hose can consist of a different plastic, e.g. polyamide, polyester, polycarbonate, TPA, Pebax, polyethylene, polypropylene or another suitable plastic.

If the wire is axially fixated inside the hose, the wire can be inserted together with the hose in the body during manufacturing of the instrument connection device. This is particularly the case if the wire is bending-resistant, e.g., in that it consists of a springy bending-resistant material, such as steel wire. The bending resistance is provided with reference to the penetration resistance, i.e., in relation to the longitudinal force applied on the wire, if it is used like a needle for penetration of the wall of the elastic body. Thereby, it is preferably at least along a length of 1 cm to 2 cm bending resistant. Preferably the wire comprises an even higher bending resistance, such that it can also penetrate through the wall of the body without bending, also in case of a free cantilevered length of at least 3 cm, at least 4 cm or at least 5 cm. During manufacturing of the gas bushing the wire can thus be used as tool for creation of the punctured hole. Preferably it is thereby only punctured through the wall once and then remains inside the punctured hole without being removed again.

If the wire is not axially fixated inside the hose, it can be inserted in the body during manufacturing of the instrument connection device first without the hose. After it has penetrated the wall of the body the hose can be threaded on the wire and inserted into the body. If the bare wire, i.e. without the hose, is penetrated through the wall, it is advantageous if it has a bending resistance that is so high that a pushing force applied on the wire outside the body is applied to the wire tip, such that it penetrates the wall. If the wire has however a lower pressure- or bending resistance, it can be inserted in the body by means of a tool that holds the wire in a distance to its proximal end, only leaving a free cantilever (sufficiently short) section in proximal direction for penetration of the wall.

The method for manufacturing the instrument connection device provides positioning of the hollow cylindrical body such that it is bent about at least 30° at a site, whereby a straight section (leg) of the body extends from the bending site. The hose with the wire end projecting therefrom is then pushed into the straight section of the body, whereby the wire penetrates the wall of the hollow cylindrical body at the bending site. If desired, the wire can be pointed or sharpened at its penetrating end, i.e., provided with a needle tip or a cutting edge. After penetration of the hollow cylindrical body the body is released, whereby it springs back in its elongated position. Thus, the gas junction for the instrument connection device is completed. The method can also be particularly carried out, if the body is angled for penetration more than 30°, e.g., 40°, 60°, 90°, 100°, 110°, 120° or more.

After penetration of the wall of the flexible hollow cylindrical body the wire can be slightly heated to create an adhesive bond at the punctured hole between the material of the hollow cylindrical body and the surface of the wire. This is however optional and depends on the material characteristics of the hollow cylindrical body.

### BRIEF DESCRIPTION OF THE DRAWINGS

Further particulars relating to advantageous details of embodiments of the invention are derived from the claims, as well as the drawing with the following figures that forms part of the specification:

FIG. 1 an instrument having an instrument connection device in accordance with an embodiment of the invention in a perspective schematic illustration,

FIG. 2 the instrument connection device according to FIG. 1 in opened illustration sectioned in part,

FIG. 3 the gas junction of the instrument connection device according to FIG. 2 in opened, partly cut illustration,

FIG. 4 a part of the wall of the hollow cylindrical body of the gas junction having a punctured hole,

FIG. 5 the section of the wall according to FIG. 4 having a punctured hole into which a wire is inserted,

FIG. 6 the instrument connection device during creation of the punctured hole in a partly longitudinally cut schematic illustration,

FIG. 7 the instrument connection device according to FIG. 6 after creation of a wire passage for the wire,

FIG. 8 a modified embodiment of the instrument connection device similar to FIG. 2, however, having a gas filter.

### DETAILED DESCRIPTION

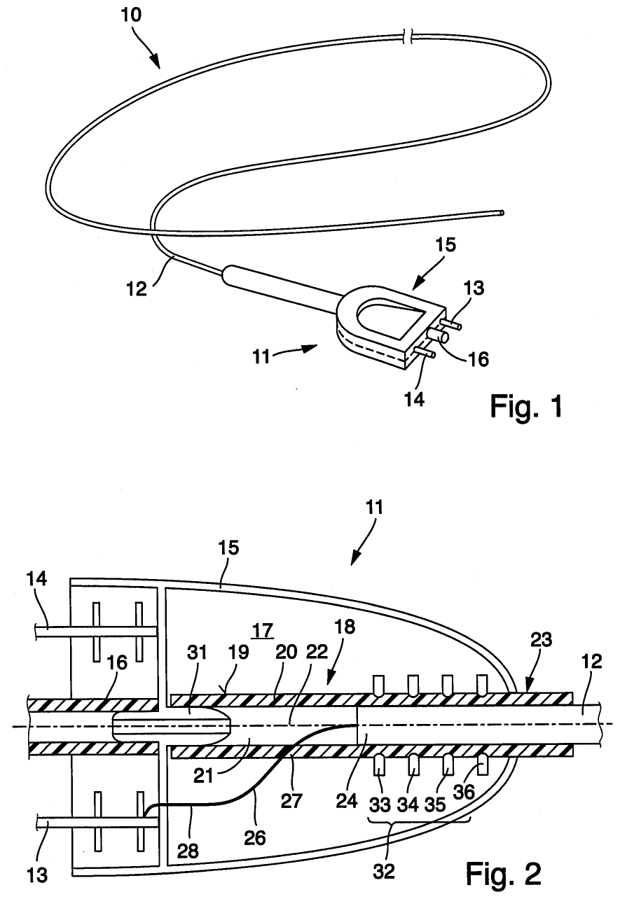

FIG. 1 illustrates an instrument 10 in form of a flexible probe, as it is suitable for endoscopic treatment of human or animal patients, for example. The instrument 10 illustrates the invention only by way of example. Embodiments of the invention can be also used with instruments in different configuration, e.g., with instruments for open surgical use or with laparoscopic instruments. It is, however, common to all such instruments that a hose 12 extends away in distal direction from an instrument connection device 11, the hose being a supply hose for instrument 10 itself, or, as illustrated in FIG. 1, being part of the instrument itself. In the example according to FIG. 1, hose 12 forms a proximal end of the instrument. In other instruments hose 12 is a supply hose that does not have to be necessarily considered as part of the instrument.

The instrument connection device 11 serves for supply of the instrument 10 with a gaseous medium, such as argon, another inert gas, a reactive gas or also a liquid as well as with voltage and/or current. The instrument illustrated in FIG. 1 by way of example is an argon plasma probe that is supplied with argon and electrical alternating current for operation. The instrument connection device 11 is, however, also suitable for other instruments, to which electrical power is supplied via an electrical line and to which a gas (or a liquid) is supplied via a hose lumen. Embodiments of the invention are particularly suitable for such instruments in which the electrical line is located inside the hose 12.

The instrument connection device 11 comprises at least one pin-shaped or otherwise configured electrical contact 13 for current or voltage supply, for example, and as necessary one or more additional electrical contacts 14. The electrical contacts 13, 14 can be pin contacts that are held parallel to one another in a connector housing 15.

The instrument connection device 11 also comprises a gas connection connector 16, which can be configured, for example, by a flexible hose-like connector piece that is in proximity of the contact pins 13, 14, e.g., between them.

FIG. 2 illustrates the configuration of the instrument connection device 11 with opened connector housing 15. The illustrated housing shell of connector housing 15 comprises the electrical contacts 13, 14 that are pin-shaped here, as well as the gas connection piece 16, all being immovably held in or on the connector housing 15. The gas connection piece 16 can be configured as flexible sleeve. The connector housing 15 in addition surrounds an interior 17 in which a gas junction 18 is arranged. This gas junction 18 serves to join the current supply and the gas supply of instrument 10. For this purpose the gas junction 18 comprises a hollow cylindrical hose-like body 19 having a flexible wall 20. The body 19 surrounds a through channel 21, the longitudinal center axis 22 of which extends preferably straight (stretched).

The wall 20 consists of a flexible, spring elastic plastic, preferably silicone plastic. The proximal end of the gas junction 18 is in fluid connection with the gas connection piece 16. For this purpose, a respective housing structure can be provided (e.g. a fluid connector 31 configured in the type of a plug fitting).

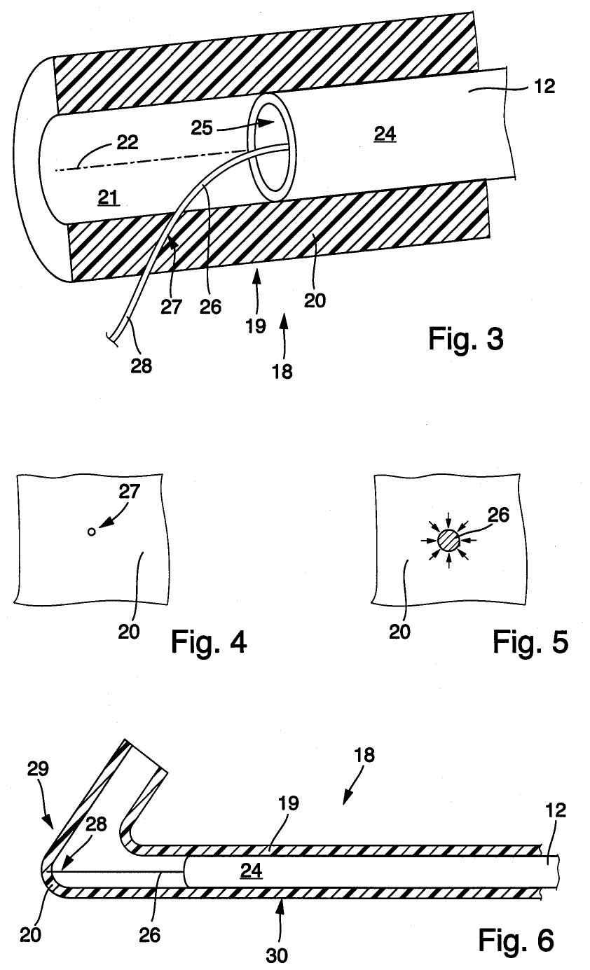

In the distal end 23 of body 20 the proximal end 24 of hose 12 is inserted. It surrounds at least one lumen 25, as particularly apparent from FIG. 3, that preferably extends from the proximal end 24 of hose 12 up to the distal end of hose 12 or also of the instrument 10. The hose 12 can also have multiple lumen extending parallel to one another over the length of the hose.

The proximal end 24 of hose 12 is inserted without play and thus abuts against the wall 20 of flexible body 19 tightly and without gap inside the through channel 21. Thereby a gas-tight connection between the through channel 21 and the lumen 25 is created.

Inside lumen 25 a wire 26 is arranged that projects out of the proximal end 24 of hose 12 and intersects the wall 20 at a punctured hole 27. Alternatively, the wire can be embedded in the plastic material of hose 12. If hose 12 has multiple lumen, the wire 26 can also be arranged in a manner extending through one of the lumen or through the material of the hose 12.

The wire 26 is preferably a bending-resistant wire, as for example a spring steel wire or a wire made of another springy bending-resistant material. The bending resistance of wire 26 is preferably so high that the wire 26, having a free cantilever length of 1 cm to 2 cm, can penetrate wall 20, if it is moved toward the latter. This is preferably also the case, if the face end of the wire 26 has not been specifically sharpened, but only comprises a shear or fractured surface. Thus, it is also clear that the expression of bending resistance depends on the penetration strength of wall 20 and thus on the material characteristic of wall 20, their thickness as well as the selected free cantilever length of wire 26. Spring steel wire having a diameter of 0.1 mm to 0.2 mm is, however, sufficiently bending resistant with usual flexible silicone materials and wall thicknesses up to multiple millimeters in case of cantilever length of up to 2 cm or more. The method according to embodiments of the invention can be carried out particularly easily by means of a wire that is so angle- and bending-resistant that it also allows a free cantilever length of at least 3, at least 4 or at least 5 cm.

The wire 26 can be uniformly configured over its entire length extending through the lumen 25 or can have joints inside or outside of gas junction 18 and thus can consist of different materials in sections. In addition, wire 26 can be surface-coated, e.g., can completely or partly comprise a silver coating or another metal coating, e.g., a copper coating. It is also possible to provide the wire (e.g., bare steel wire or metal-coated steel wire) in addition with a non-metallic coating that immovably adheres to its surface, particularly a thermo-plastic coating. The non-metallic coating can extend over the entire length of the wire or alternatively also only over a section of its length, e.g., over the part projecting out of hose 12.

The punctured hole 27 is preferably punctured by wire 26 itself and thus created without material removal. If the wire 26 would be removed from punctured hole 27, it would at least nearly or also completely close again under elastic release of wall 20, as shown in FIG. 4. However, if the wire 26 is present in the punctured hole 27, as shown in FIG. 5, the wall 20 abuts under pretension against the wire 26 and thus seals there. In doing so, the wall of punctured hole 27 forms with wire 26 a gas-tight wire passage.

Preferably the punctured hole 27 is arranged in an acute angle relative to the longitudinal axis 22 of the through channel 21. The angle limited between the punctured hole 27 and the longitudinal axis 22 is thereby less than or equal to 90°, preferably less than 80°, further preferably less than 70° and best less than 60°. On the other hand, the angle is larger than 10°, preferably larger than 20°, better larger than 30° and preferably larger than 40°. With this dimensioning, a simple producibility and concurrently a good sealing tightness of the wire passage are achieved.

The wire 36 can be loosely placed inside lumen 25, such that no axially rigid connection exists between the wire 26 and the hose 12. The wire 26 can, however, also be axially immovably connected with the hose 12, e.g., by means of respectively arranged holders or by means of structures of the hose 12 inside lumen 25.

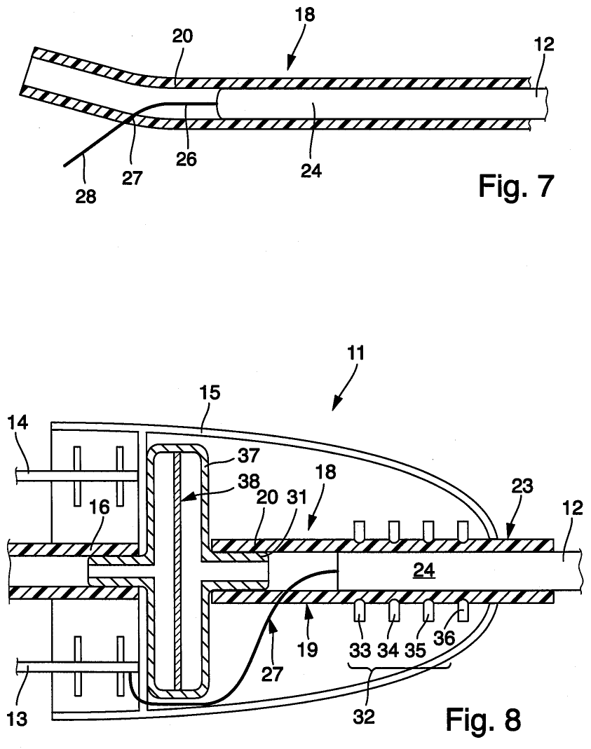

For further illustration of embodiments of the invention, FIGS. 6 and 7 show manufacturing steps of gas junction 18.

For manufacturing of gas junction 18, first the hollow cylindrical body 19 and the wire 26 are provided. The wire 26 can be provided as bare wire or in case it is connected with hose 12, also together with hose 12, whereby however the proximal end 28 of wire 26 projects from the proximal end 24 of hose 12 about a desired amount, e.g., of 1 cm to 2 cm or also of multiple centimeters.

The body 19 is now brought into the angled shape illustrated in FIG. 6, having a bending site 29 at which the hose-like body 19 is angled about 30° or more. An angle of more than 90° is preferred. At least one straight leg 30 extends from the bending location 29 that is provided for locating the proximal end 24 of hose 12 therein.

The wire 26 is now inserted into leg 30, such that its proximal end 28 hits this location of the wall 20, preferably at an approximately right angle. Further forwarding of wire 26 has the effect that wire 26 penetrates through wall 20 under creation of punctured hole 27. Concurrently or subsequently hose 12 is inserted with its proximal end 24 into leg 30.

After having carried out this procedure the gas junction 18 completed to this extent is removed from a holding device such that the bending site 29 can release and stretch again. The gas junction 18 then takes approximately the shape illustrated in FIG. 7. Depending on the spring constant of wire 26 and wall 20, the latter is again completely hollow cylindrically straight or as illustrated in FIG. 7, still slightly angled. However, the wire 26 is inserted in the punctured hole 27 in a fluid-tight manner.

In the next step gas junction 18 can now be installed connector housing 15. For this purpose, as obvious from FIG. 2, body 19 is pushed with its proximal end onto the fluid connector 31 that can be configured as housing structure and establishes the fluid connection to the gas connection piece 16. In addition, the proximal end 28 of wire 26 can be electrically and mechanically connected with a contact 13 or 14 (or with both), e.g. soldered, welded, crimped or otherwise connected. In addition, gas junction 18 is inserted into a clamping structure 32, at least where body 19 has included proximal end 24 of hose 12. The clamping structure 32 can consist of one or more wall sections 33, 34, 35, 36 that can be an inseparable part of the housing shell of connector housing 15 and respectively comprise a U-shaped cutout, the clearance of which is slightly less than the outer diameter of body 19. The latter is deformed radially inwardly by means of the wall sections 33 to 36, such that the proximal end 24 of hose 12 is clamped within clamping device 32 inside body 19. Concurrently the body 19 is secured inside housing 15 in a tensile-resistant manner. Preferably the clamping structure is elastically movable in axial direction. The web width of wall sections 33, 34, 35, 36 is less than ⅕, 1/7 or 1/10 of the outer diameter of the flexible body. In doing so, an improved fixation of gas junction inside the half shell results.

In FIG. 2 only a lower housing shell is illustrated. The removed upper housing shell can comprise such wall sections as clamping structure that extend between the wall sections 33 to 36 shown in FIG. 2 and thus complement the clamping of hose 12 inside gas junction 18.

FIG. 8 illustrates an embodiment of the invention in which the fluid connector 31 is part of a filter housing with a gas filter 38 provided therein. The gas filter 38 can be a fine-pored body that blocks the transfer of contaminations from the supplying apparatus into the instrument 10 as well as the retransfer of contaminations from the instrument 10 into the supplying apparatus. Apart therefrom, the description given with reference to FIGS. 1-7 above applies accordingly based on the same reference signs.

An instrument connection device 11 according to embodiments of the invention comprises a flexible hose-like body 19 having a flexible wall 20 in which the proximal end 24 of a hose 12 is inserted that is part of the instrument 10 or leads up to the latter. The hose 12 comprises a lumen 25 that extends longitudinally through the hose 12. In addition, a wire 26 is arranged inside hose 12, e.g. inside lumen 25. It is guided through a punctured hole 27 that the wire 26 produces itself during penetration of wall 20. The punctured hole 27 is preferably straight and guides obliquely, i.e. inclined to the radial as well as also to the longitudinal center axis, through the wall 20.

1. An instrument connection device comprising:
a gas junction comprising a hollow cylindrical body having a flexible wall having a punctured hole and configured to limit a through channel;
a hose comprising at least one lumen and arranged to abut the wall inside the body, the at least one lumen having a first end extending in proximal direction into the hollow cylindrical body; and
a wire arranged inside the hose, projecting proximally from the hose and extending through the punctured hole.

2. The instrument connection device according to claim 1,
wherein the punctured hole is configured to be elastically widened by the wire.

3. The instrument connection device according to claim 1,
wherein the flexible wall abuts in an elastically tensioned manner against the wire at the punctured hole.

4. The instrument connection device according to claim 1, wherein the wire extends through the punctured hole without a gasket.

5. The instrument connection device according to claim 1, wherein the punctured hole is one of oriented at a right angle relative to the wall or inclined relative to the wall.

6. The instrument connection device according to claim 1, wherein the punctured hole is arranged at an angle relative to a longitudinal direction the through channel that is less than 90°.

7. The instrument connection device according to claim 6, wherein the punctured hole is arranged at an angle relative to the longitudinal direction that is larger than 10°.

8. The instrument connection device according to claim 1, wherein the punctured hole is arranged at an angle relative to the longitudinal direction that is larger than 10°.

9. The instrument connection device according to claim 1, wherein the hollow cylindrical body has a flexibility that is greater than a flexibility of the hose.

10. The instrument connection device according to claim 1, wherein the hose is held inside the hollow cylindrical body in a friction-fit manner.

11. The instrument connection device according to claim 1, wherein the hollow cylindrical body comprises a silicone plastic.

12. The instrument connection device according to claim 1, wherein the hose comprises at least one of a polyamide, a polyester, a polycarbonate, TPA, Pebax, polypropylene and a polyethylene.

13. The instrument connection device according to claim 1, wherein the wire is fixed inside the hose.

14. The instrument connection device according to claim 1, wherein the wire comprises a steel wire.

15. The instrument connection device according to claim 1, wherein the wire comprises a springy bending-resistant material.

16. An instrument having an instrument connection device according to claim 1.

17. A method for manufacturing an instrument connection device according to claim 1, the method comprising:
first providing the hose with the wire projecting from its proximal end and the hollow cylindrical body;
thereafter elastically bending the hollow cylindrical body at a site such that at least one straight section of the hollow cylindrical body extends from the site;
thereafter inserting the proximal end of the wire into the straight section such that the wire penetrates body at the bent site; and
thereafter transferring the hollow cylindrical body back into a released elongate shape.
